(19) United States
(12) Patent Application Publication
(10) Pub. No.: US 2013/0131653 A1
(43) Pub. Date: May 23, 2013

(54) Ophthalmological laser method and apparatus

(71) Applicant: Cheng-Hao Huang, Orlando, FL (US)

(72) Inventor: Cheng-Hao Huang, Orlando, FL (US)

(73) Assignee: Excelsius Medical, Inc.

(21) Appl. No.: 13/373,591

(22) Filed: Nov. 22, 2011

(51) Int. Cl. A61F 9/01; A61F 9/009

(52) U.S. Cl. 606/5

(57) ABSTRACT

The present invention relates to a femtosecond laser ophthalmological system and especially to the use of a femtosecond laser to create a flap for LASIK refractive surgery or for other applications that require removal of corneal or lens tissue at specific locations such as in corneal transplants, stromal tunnels, corneal lenticular extraction and cataract surgery. The output of a femtosecond laser is scanned into overlapping circles of laser pulses which are then moved in an overlapping trajectory on a patient's eye to ablate the eye tissue in a predetermined pattern.

### BACKGROUND OF THE INVENTION

The present invention relates to a femtosecond laser ophthalmological system and especially to the use of a femtosecond laser to create a flap for LASIK refractive surgery or for other applications that require removal of corneal and lens tissue at specific locations such as in corneal transplants, stromal tunnels, corneal lenticular extraction and cataract surgery.

Ametropia such as myopia (short-sightedness), hyperopia (long-sightedness or far-sightedness) or astigmatism can nowadays be permanently corrected by refractive surgical treatment. Refractive surgical treatments are operations on the eye which change the optical refractive power of the eye with the aim of bringing it as close to a desired value as possible. One of the most important methods in refractive surgery is laser-assisted in situ keratomileusis (LASIK) in which corneal tissue is removed with the aid of a computer-controlled excimer laser after a corneal flap has previously been partially severed and folded away. In order to produce the corneal flap, use is made of mechanical microkeratomes in which a driven scalpel cuts the corneal flap. Such corneal flaps can now also be cut with the aid of strongly focused femtosecond laser pluses, which have pulse widths of 100 fs to 1,000 fs (1 fs=10⁻¹⁵ s). The risks existing during use of a mechanically oscillating scalpel are avoided by the use of a femtosecond laser. Such a system is marketed, for example, by IntraLase Corp, in Irvine, Calif., USA under the name of Pulsion FS Laser. The overall size of the known systems, having femtosecond lasers is comparable to the overall size of an excimer laser system, the disadvantage being that the space required for the excimer laser system is required once again in the treatment room for the femtosecond laser system. In addition, after cutting the corneal flap with the femtosecond laser system the patient must be transferred to the excimer laser. The overall size of the femtosecond laser system is determined by the light source used, the scanner technology and the attendant beam guidance system.

The femtosecond laser beam is focused with a large lens system by means of beam-deflecting optical elements onto the tissue areas of the eye to be separated. For design reasons, the maximum achievable numerical aperture (NA, proportional to half the size of the sine of the aperture angle of the lens) of the focusing optical system is limited in this case (typically NA=0.2-0.3). For a given work area, for example the entire cornea, the scanning optics (typically F-Theta optics) requires a minimum working distance. In conjunction with the required movement and the achievable size of the beam-deflecting optical elements, the working distance determines a design limit for the diameter of the scanning optics. A further upper design limit for the diameter follows from instances of collisions with body parts (eyebrows, nose). Even when using a large diameter, it is possible to illuminate only a subarea of the optics with a scanning laser beam. The result of this is an upper design limit for the effective useful numerical aperture of the optics. High apertures are desirable because with a high numerical aperture (NA) it is possible to produce small focal points, and thus a smaller cutting zone per pulse. Less gas is produced per pulse in smaller cutting zones than in larger cutting zones. More precise cuts can be made by means of smaller gas bubbles since the cutting zones are not substantially deformed by the internal gas pressure. In addition, a high numerical aperture requires disproportionately less energy per pulse to make a cut. With lower energy, there is also a reduction in the size of the cavitation bubbles produced by the laser pulse which has a positive effect on the cutting quality. Also, the cavitation bubble is more quickly absorbed. Furthermore, the retina is subject to less stress from the more strongly diverging beams downstream of the focal point with high numerical apertures. A further advantage is that a high numerical aperture lens's highly localized focusing capability has less effect in the vicinity of the focus point.

U.S. Pat. No. 5,549,632 describes an ophthalmological apparatus having a laser source for the breakdown of eye tissue, which can be used for cutting corneal flaps. The apparatus in accordance with this Patent comprises a laser source and a projection head, optically connected to the laser source, in a housing separate from the laser source. The patent also comprises beam control means which control the beam path of the laser pulses emitted by the laser source such that points in a reference frame are fixed relative to the laser source and are imaged via an optical connection onto corresponding points in a reference frame fixed relative to the projection head. The optical connection is designed as an articulated mirror arm so the laser pulses deflected by the beam control means can be imaged relative to the reference frame of the hand-held appliance. The connection of the projection head to an application plate which can be permanently connected to the eye means that the fixed reference frame of the projection head is permanently imaged onto the application plate and thus onto the eye. In this patent the laser pulses are fed to desired positions of the eye by using the beam control means to control the position of the pulsed laser beam relative to the application plate and to image it onto the eye via the optical connection and the optical projection system of the projection head. For example, in order to carry out cuts of 5 to 15 mm in length, the optical projection system of the projection head must have optical lenses whose diameter is greater than the diameter of the eyeball. A projection head of such large dimension will cover the view onto the eye to be treated. Furthermore, the numerical aperture of the apparatus is small as may be seen from the relatively small convergence of the beams. Large lens systems also have the disadvantage of causing devices to become heavy and unwieldy, thus complicating the manual holding application.

In U.S. Patent Application Publication No. US 2004/0254568 to Rathjen, an ophthalmological apparatus for the breakdown of eye tissue is shown which includes an application head which can be mounted on the eye and is provided with a light projector. The light projector is moved with the aid of movement drivers in order to bring the focal point to the desired site for tissue breakdown. According to this Publication the fine movement of the focal point can additionally be superimposed on the translatory movements of the light projector by means of optical microscans. In order, however, to be able to deflect grossly expanded, non-convergent light beams advantageous for cutting, there is a need for relatively large mirrors (for example 14 mm) which must be tilted by relatively large angels (for example 4 degrees). Such large scanner systems do not however, permit high speeds given the present prior art, and cannot be of compact design. If the aim is to avoid the above-named disadvantages of a large overall size of the application head, and if the aim is to prevent the same site in the eye tissue from being repeatedly hit by the laser pulses, it is possible to make use of the application head of simple optical microscans for low pulse rates. However, low pulse rates means an operation procedure that is slowed down and thus less stable.

In Rathjen et al, U.S. Pat. No. 7,621,637, an ophthalmological apparatus for the breakdown of eye tissue does not have some of the disadvantages of the prior art. This ophthalmological apparatus has a base station having a light source for generating light pulses, and an application head which can be mounted on an eye. The application head is provided with a light projector for the focused projection of the light pulses for punctiform breakdown of eye tissue, and has movement drivers for moving the light projector in a free direction and in a first scanning direction. The application head has, for example, a contact body which can be mounted on the eye, is at least partly transparent and is configured and arranged so that it sets a region of the eye whose contact is made in the mounted state in a fashion equidistant from a working surface. The application head also has, for example, fastening means for fixing the application head at the eye by low pressure.

In the ophthalmological apparatus described in U.S. Pat. No. 7,621,637 a rotating mirror is used in the base station to create a line laser pulse scanning pattern. One disadvantage in this system is that when the laser light pulses scan a line across the focusing lens, the incident angle of the beam varies and the resulting focal points vary in distance from the focal plane. A method of repositioning these points during the scan so that they are all on the focal plane is a primary concern of another Rathjen U.S. Pat. No. 7,597,444. A second disadvantage of this patent is that the laser pulse line scanning pattern, after having been transmitted through the delivery arm, has to be precisely aligned to be perpendicular with the trajectory of the slow translation motor, otherwise the final pattern on the eye created by the slow motor would be distorted. This patent uses a rotation element to compensate for the rotation of the light pulse line scanning direction caused by the optical transmission system of the articulated arm. A third disadvantage of this apparatus is that the width of the scanned subarea may vary if the trajectory is not linear. This limits the trajectories available upon the target, thus limiting the possible applications especially when a 3-dimensional curvature is required inside the cornea or the lens tissue. In both cases, regular and irregular topologies can be ablated more efficiently by using concentric or spiral trajectories.

Rathjen is also an inventor on U.S. Patent Application Publication No. US 2011/0205492 for an Ophthalmological Device which uses a femtosecond laser in a controllable optical correction element for the variable modulation of the wavefront of deflected femtosecond laser pulses.

In the present invention an ophthalmological apparatus has a femtosecond laser whose beam is enlarged and deflected by an XY galvanometer device in an overlapping circular scanning pattern in a base station. An optical transmission path transmits the circular scanning pattern of laser light pulses from the base station to a remote application head having an XYZ piezo positioning stage supported focusing lens which drives the overlapping circular scanning pattern, in an overlapping manner, in a predetermined trajectory onto a patient's eye to ablate the area being scanned. The overlapping circular scanning pattern of laser beam spots impinges on the focusing lens around the center axis of the focusing lens at a constant distance from the center axis making the focal points all in the focal plane without compensating optics as required by the prior art. Furthermore, the circular scanning pattern is automatically aligned perpendicularly with the trajectory of the XY piezo positioning stage since the overlapping circular scanning pattern is not sensitive to any rotational factors induced by the mirror arm. The resultant ablation patterns are of a consistent line width that is not sensitive to the direction of the trajectory. This provides flexibility for different trajectories such as line patterns, concentric circle patterns and spiral patterns.

### SUMMARY OF THE INVENTION

A method of ablating eye tissue includes generating a pulsed laser beam from a femtosecond laser and applying the generated laser beam to a laser scanner to generate an overlapping, generally circular scanning pattern of laser pulses. The overlapping circular scanning pattern of laser pulses is applied co-axially to an XYZ piezo positioning stage supported focusing lens which focuses the overlapping circular pattern of laser pulses on to a focal plane such as a patient's eye. The overlapping circular pattern of laser pulses are scanned in a generally overlapping trajectory onto a patient's eye to ablate a predetermined pattern of eye tissue in the patient's eye. A patient's eye is thus ablated in a predetermined overlapping trajectory with a controlled focus of a femtosecond laser with an overlapping circular scanning pattern of laser pulses. Such overlapping trajectories include concentric circles, parallel lines, spiral patterns and other regular and irregular topologies.

The ophthalmological apparatus has a main cabinet and a hand piece connected thereto with an articulated arm. A femtosecond laser is positioned in the main cabinet and has a laser beam output of laser pulses. A laser beam enlarger is positioned to enlarge the femtosecond laser beam laser pulses. An XY galvanometer laser scanner is positioned in the main cabinet for scanning the laser beam into an overlapping circular scanning pattern of laser pulses. An XYZ piezo positioning stage is located in the hand piece that supports a focusing lens and is positioned to receive the overlapping circular scanning pattern of laser pulses from the laser scanner which are centered on the center axis of the focusing lens. The focusing lens is positioned to focus the overlapping circular scanning pattern of laser pulses onto a patient's eye while scanning the overlapping circular scanning pattern of laser pulses in a predetermined overlapping trajectory with the XYZ piezo positioning stage to ablate a predetermined subarea of tissue on a patient's eye. Thus a patient's eye has a predetermined area thereon ablated by overlapping circles of laser pulses.

### BRIEF DESCRIPTION OF THE DRAWINGS

The accompanying drawings, which are included to provide further understanding of the invention and are incorporated in and constitute a part of the specification, illustrate an embodiment of the invention and together with the description serve to explain the principles of the invention.

In the drawings:

FIG. 1 shows a block diagram of an ophthalmologic laser surgery apparatus in accordance with the present invention;

FIG. 2 is an overlapping circular scanning pattern of laser beam spots on a focusing lens;

FIG. 3 is an overlapping circular scanning pattern of laser beam spots consistently focused on a focal plane;

FIG. 4 shows a concentric circle trajectory of an overlapping circular scanning pattern of laser beam spots;

FIG. 5 shows a parallel line trajectory of an overlapping circular scanning pattern of laser beam spots; and

FIG. 6 shows a spiral trajectory of an overlapping circular scanning pattern of laser beam spots.

### DETAILED DESCRIPTION OF EXEMPLARY EMBODIMENT

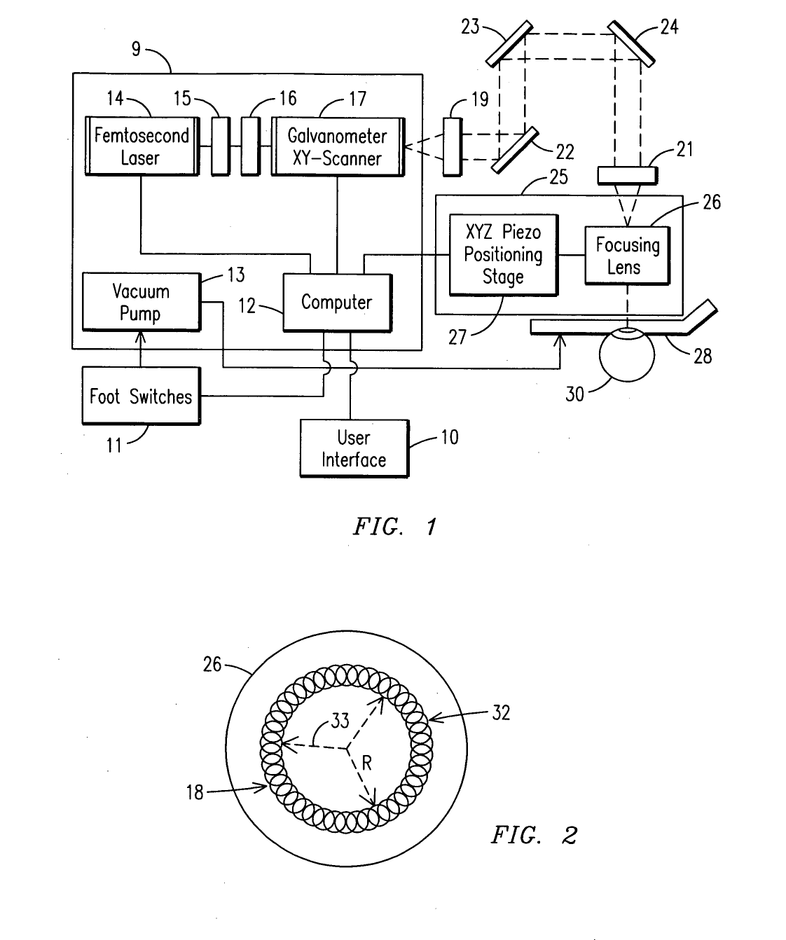

A laser ophthalmological surgery apparatus in accordance with the present invention as seen in the drawings includes a user interface connected to a main cabinet 9 in FIG. 1. Within the main cabinet, laser pulses are generated that are deflected by XY-galvanometers (galvos)17 to create a repetitive overlapping circular scanning pattern 18 in FIG. 2. The overlapping circular scanning pattern 18 of laser beam spots 32 passes through a relay lens 19 and mirror arm to a remote hand piece where it has the same characteristics and properties as the output scanned galvos 17. Inside the hand piece, a focusing lens 26 is used to reduce the size of the laser beam spots 32 and the size of the overlapping circular scanning pattern 18. An XYZ piezo positioning stage 27 is used to move the overlapping circular scanning pattern 18 according to a predetermined trajectory on the cornea or lens. The hand piece 25 is attached to the eye 30 via a suction ring 28. The suction ring is connected to the hand piece 25 by a translation track. Since the suction ring 28 is completely clear when viewed under the microscope, the alignment is more easily performed.

Following the light path in greater detail, the light pulse generator is a femtosecond laser (wavelength 340 nm to 2000 nm, pulse width less than 1000 femtosecond and a pulse repetition rate between 10 KHz to 1 MHz). The laser beam spot size is enlarged by a beam expander 15 and is then blocked by the shutter 16 until the foot switch 11 is depressed. While the foot switch is pressed, the beam is allowed to continue to the XY-galvanometer 17 where it is deflected to create a repetitive overlapping circular scanning pattern 18 as shown in FIG. 2. The overlapping circular scanning pattern 18 may have a repetition rate up to 5 Khz. The laser beam overlapping circular pattern 18 passes through relay lens 19 and through the mirror arm having mirrors 23 and 24 therein and into the relay lens 21. The purpose of the lenses and mirrors is so that the overlapping circular scanning pattern 18 enters the hand piece 25 with the same characteristics and properties as the output scanned from the galvos 17.

Inside the hand piece 25, a compact, low F-number (high NA) focusing lens 26 is mounted on an XYZ piezo positioning stage 27 which reduces the spot size of the laser beam spots to less than 3 microns and the size of the overlapping circular scanning pattern 18 to a diameter of less than 500 microns. The focusing lens 26 has a smaller size and has a higher focusing capability than the F-Theta lens most Often used in prior art. The higher focusing capability can generate a small laser beam spot size which reduces the required energy based on the same energy density. Reduced energy levels form smaller cavitation bubbles that are more quickly absorbed by the surrounding tissue. This also means the acoustic wave impact caused by the photodisruption is also reduced. Furthermore, the smaller lens size can be incorporated into a small hand piece that makes it easier to integrate this laser apparatus with existing LASIK surgical instruments.

The hand piece 25 is attached to the eye 30 with a disposable suction ring 28. First the disposable suction ring 28 is connected to the hand piece 25 by a translation track. The suction ring is placed on the eye and aligned using a microscope. Once aligned, the hand piece is manually pushed into the proper position as dictated by the suction ring.

The ablation patterns generated by this laser apparatus are based on a series of overlapping circles rather than a series of short line segments used by prior art. Therefore, the resultant ablation patterns are of a consistent line width that is not sensitive to the direction of trajectory. This will provide the flexibility of different trajectories such as parallel line patterns, concentric circular patterns, spiral patterns, or other regular or irregular topology patterns. Also, the laser beam overlapping circular scanning pattern is always a consistent radius from the center of the focusing lens making the focal points all on the focal plane without any compensation optics as required by prior art. Furthermore, the laser beam overlapping circular scanning pattern 18 is automatically aligned perpendicularly with the trajectory of the XY piezo positioning stage 27 since the overlapping circular pattern is not sensitive to any rotational factors induced by the mirror arm.

Referring to the drawings, a femtosecond laser ophthalmological apparatus is illustrated in FIG. 1. The foot switches 11 and the user interface 10 are connected to the computer 12 inside the main cabinet 9. The main cabinet 9 contains a femtosecond laser 14 which generates the laser light pulses. The laser beam passes through the beam expander 15 used to enlarge the laser beam spot size. The laser beam is blocked by the shutter 16 until a foot switch 11 is depressed. When the foot switch 11 is pressed the laser beam is impinged upon the XY galvanometer or galvos 17. The galvanometer 17 deflects the beam into an overlapping circular scanning pattern 18 as seen in FIG. 2. The laser beam overlapping circular scanning pattern 18 then passes through a system of relay lenses 19 and 21 and is deflected by mirrors 22, 23 and 24 which connect the laser beam overlapping circular scanning pattern 18 from the main cabinet 9 to a remote hand piece 25 with an articulated arm holding the mirrors and relay lens. The hand piece 25 contains a focusing lens 26 which is mounted to an XYZ piezo positioning stage 27. The laser beam overlapping circular scanning pattern 18 is focused by the focusing lens 26 to form the final trajectory set by the XYZ piezo positioning stage 27.

A disposable suction ring 28 is connected to the Hand Piece 25 by a translation track and positioned to be secured to a patient's eye 30 by the low pressure created and maintained by a vacuum pump 31. The laser beam passes through the disposable suction ring 28 into the cornea of the eye 30.

FIG. 2 illustrates the overlapping circular scanning pattern 18 of the laser beam spots 32 created by the XY-galvanometer 17 which is impinged upon the focusing lens 26. The radius 33 of the overlapping circular scanning pattern is illustrated in FIG. 2. In FIG. 2, the circle 18 of laser beam spots 32 can be seen to pass through the focusing lens 26 on the same radius from lens center with the same refraction of the lens for the entire circle so that the refraction of the laser beam spots passing through the lens is the same for each laser beam spot 32. That is, since the scan is done in a circular pattern on the focusing lens, the incident angle of the beam is always consistent, with a specific radius from the center axis of the lens, and the focal points are all on the focal plane.

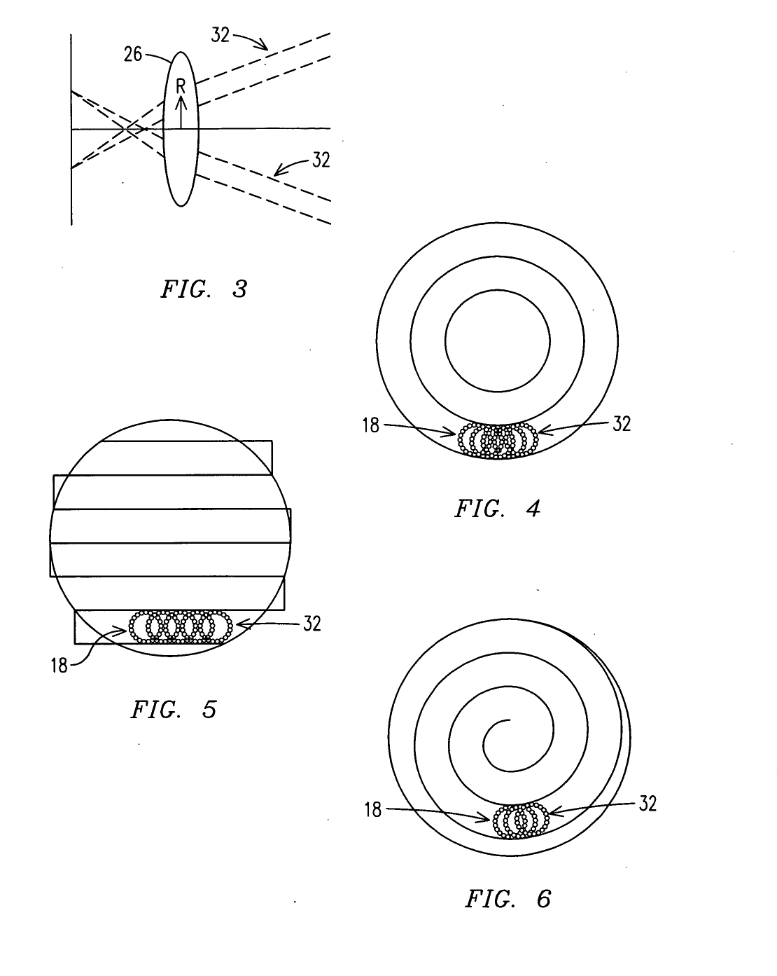

In FIG. 3 the circular pattern 18 of the laser beam spots 32 are kept in continuous focus by the focusing lens 26 onto the focal plane F.

FIG. 4 illustrates one of the many possible trajectories of the overlapping circular scanning pattern 18 of laser beam spots 32. In FIG. 4 the trajectory of overlapping circular scanning pattern 18 of laser beam spots 32 is moved around in concentric circles to ablate the area being focused on under control of the computer 12.

FIG. 5 illustrates a trajectory of overlapping circular scanning pattern 18 of laser beam spots 32 in parallel lines.

FIG. 6 illustrates a trajectory of overlapping circular scanning pattern 18 of laser beam spots 32 in a spiral pattern.

The laser beam ophthalmological surgery method of the present invention includes the steps of generating a laser beam which is scanned in a series of overlapping circles with an XY-galvanometer 17 or other scanning device that creates a repetitive overlapping circle pattern 18. The beam is relayed through an optical system to a remote location where it has the same characteristics and properties as the output scanned from the XY galvanometer 17. A focusing lens 26 is then used to reduce the spot size of the laser beam (to around 0.002 mm) and the size of the scanned circles (to a diameter range between 0.05 mm to 0.5 mm on the target plane). An XYZ piezo positioning stage 27 is used to move the laser beam according to a predetermined ablation trajectory in the cornea or lens. This method has a simple focusing lens 26 which is smaller and has a higher focusing capability than the F-Theta lens most often used in existing systems. The higher focusing capability can generate smaller laser beam spots which reduces the required energy based on the same energy density. Reduced energy levels form smaller cavitation bubbles that are more quickly absorbed by the surrounding tissue. Also the acoustic wave impact caused by the photo disruption is reduced. The smaller lens 26 can, advantageously, be incorporated into a small hand piece 25 that makes it easier to integrate the laser apparatus with existing LASIK surgical instruments. The ablation patterns generated by the present invention are based on a series of overlapping circles 18 rather than a series of short line segments used in existing systems so that the resultant ablation patterns are of a consistent line width (diameters ranging from 0.05 mm to 0.5 mm on the target plane) that is not sensitive to the direction of motion. This provides flexibility for a wide range of applications including corneal transplants, stromal tunnels, corneal lenticular extraction and cataract surgery.

It should be clear at this time that a femtosecond laser eye surgery method and apparatus have been provided which advantageously allows an overlapping circular scanning pattern of laser beam spots while maintaining a consistent focusing plane for a predetermined trajectory. However, it should be clear that the present invention is not to be considered as limited to the forms shown which are to be considered illustrative rather than restrictive.

I claim:

1. A method of ablating eye tissue comprising the steps of:
generating a laser beam from a femtosecond laser;
applying the generated laser beam to a laser scanner to generate an overlapping, generally circular scanning pattern of laser pulses;
selecting an XYZ piezo positioning stage supported focusing lens;
applying the overlapping, generally circular scanning pattern of laser pulses generated by said laser scanner onto the focusing lens centered around the center axis of the focusing lens;
focusing the overlapping, generally circular scanning pattern of laser pulses onto a patient's eye; and
scanning the overlapping, generally circular scanning pattern of laser pulses in a generally overlapping trajectory onto the patient's eye to ablate a predetermined pattern of eye tissue on the patient's eye;
whereby a patient's eye can be ablated in a predetermined trajectory with a controlled focus of a femtosecond laser with overlapping circles of laser pulses.

2. The method of ablating eye tissue in accordance with claim 1 which includes scanning the overlapping, generally circular scanning pattern of laser pulses in a trajectory of generally concentric overlapping circular laser pulses.

3. The method of ablating eye tissue in accordance with claim 1 which includes scanning the overlapping, generally circular scanning pattern of laser pulses in a trajectory of generally parallel lines of overlapping circular laser pulses.

4. The method of ablating eye tissue in accordance with claim 1 which includes scanning the overlapping, generally circular scanning pattern of laser pulses in a trajectory of generally spiral overlapping circular laser pulses.

5. The method of ablating eye tissue in accordance with claim 1 which includes scanning the femtosecond laser generated laser beam with an XY galvanometer to generate the overlapping, generally circular scanning pattern of laser pulses.

6. The method of ablating eye tissue in accordance with claim 1 including mounting said selected XYZ piezo positioning stage supported focusing lens in an adjustable hand piece.

7. The method of ablating eye tissue in accordance with claim 6 including mounting said femtosecond laser and XY galvanometer in a main cabinet connected to said adjustable hand piece with an articulated arm.

8. The method of ablating eye tissue in accordance with claim 1 including the step of selecting a beam expander for enlarging the femtosecond laser beam spot size.

9. The method of ablating eye tissue in accordance with claim 7 which includes selecting a hand piece having a suction ring for attaching said hand piece to a patient's eye.

10. The method of ablating eye tissue in accordance with claim 1 which includes scanning the overlapping, generally circular laser scanning pattern of laser pulses in a trajectory of regular or irregular topologies of overlapping circular laser pulses.

11. An ophthalmological apparatus comprising:
a main cabinet and a hand piece connected to said main cabinet with an articulated arm;
a femtosecond laser positioned in said main cabinet and having a laser beam output of laser pulses;
a laser beam pulse enlarger positioned to enlarge said femtosecond laser beam laser pulses;
a laser scanner positioned in said main cabinet for scanning said laser beam into an overlapping circular scanning pattern of laser pulses;
a XYZ piezo positioning stage located in said hand piece; and
a focusing lens mounted to said XYZ piezo positioning stage and positioned for receiving the overlapping circular scanning pattern of laser pulses from said laser scanner, centered on the center axis of said focusing lens, said focusing lens positioned to focus said overlapping circular scanning pattern of laser pulses onto a patient's eye while scanning said overlapping circular scanning pattern of laser pulses in a predetermined overlapping trajectory with said XYZ piezo positioning stage to ablate a predetermined pattern of tissue on the patient's eye;
whereby a patient's eye can have a predetermined area thereon ablated by overlapping circles of laser pulses.

12. An ophthalmological apparatus in accordance with claim 11 in which said laser scanner is an XY galvanometer scanner.

13. An ophthalmological apparatus in accordance with claim 11 in which said articulated arm has a plurality of mirrors therein.

14. An ophthalmological apparatus in accordance with claim 13 in which said articulated arm has a plurality of relay lenses therein.

15. An ophthalmological apparatus in accordance with claim 12 including a manually activated shutter mounted between said femtosecond laser and said galvanometer XY scanner.

16. An ophthalmological apparatus in accordance with claim 11 in which said hand piece has a suction ring for attaching said hand piece to a patient's eye.
